(19) United States
(12) Patent Application Publication
(10) Pub. No.: US 2013/0225947 A1
(43) Pub. Date: Aug. 29, 2013

(54) IMPLANTABLE MEDICAL DEVICE

(71) Applicant: Andreas Blomqvist, Taby (SE)

(72) Inventor: Andreas Blomqvist, Taby (SE)

(73) Assignee: ST. JUDE MEDICAL AB, JARFALLA (SE)

(21) Appl. No.: 13/881,980

(22) PCT Filed: Oct. 29, 2010
(86) PCT No.: PCT/EP10/66468

§ 371 Date: Apr. 26, 2013

(51) Int. Cl. A61B 5/0295; A61N 1/362; A61B 5/0265; A61B 5/145; A61B 5/026; A61B 5/00; A61B 5/0205

(52) U.S. Cl. CPC A61B 5/0295; A61B 5/686; A61B 5/4836; A61N 1/362; A61B 5/0205; A61B 5/14542; A61B 5/14503; A61B 5/0261; A61B 5/7275; A61B 5/7282; A61B 5/0265
USPC 600/301; 600/508; 600/479

(57) ABSTRACT

An implantable medical device comprising a coronary perfusion measurement unit is adapted to measure and determine parameters related to coronary perfusion of heart tissue. The parameters include time periods and perfusion magnitudes. The coronary perfusion measurement unit is configured to determine a time period T related to a perfusion event of a coronary vessel and including includes a reperfusion time period, where a perfusion event is defined as a decrease of coronary perfusion followed by reperfusion, and to generate a time period signal in dependence thereto. The implantable medical device further comprises a coronary flow calculation unit that is adapted to receive the time period signal and that is adapted to process the time period and to generate an ischemia risk indicating index I in dependence of the time period.

### FIELD OF THE INVENTION

The present invention relates to an implantable medical device, and a method for determining an ischemia risk, according to the preambles of the independent claims.

### BACKGROUND OF THE INVENTION

Perfusion is a physiological term that refers to the process of nutritive delivery of arterial blood to a capillary bed in the biological tissue, herein in particular in heart tissue.

For ischemic patients the normal compensatory mechanism in auto-regulating blood flow is generally decreased by the underlying cardiovascular disease (atherosclerosis). Ischemic patients are therefore very sensitive to changes in coronary perfusion and this perfusion is adversely affected by atherosclerosis. The calcification of the coronary vessels is usually a slow process and asymptomatic for a long time.

There is a need to improve the diagnosis of ischemia since early detection and subsequent treatment may limit the harmful effects and reduce complications and suffering to a minimum.

One way to detect and evaluate the severity of stenosis in coronary arteries is the so-called fractional flow reserve (FFR) method. FFR is an index for functional severity of coronary stenosis measured e.g. by a miniaturized pressure sensor arranged at the distal end of a guidewire that is inserted into the coronary artery to a suspected stenosis.

FFR is 100% specific in identifying which lesion or lesions are responsible for a patient's ischemia, enabling the interventional cardiologist's direction of coronary interventions and result assessment for improved treatment outcomes. This technique has proved efficacy and is a way of the future in evaluating which stenosis to stent and which to leave alone. However, the FFR-method is both time-consuming and requires coronary artery catheterization.

In the art several methods directed to detection of ischemia are known which will be briefly described in the following.

U.S. Pat. No. 7,460,900 discloses a method and apparatus for detecting ischemia using changes in QRS morphology. This is achieved by determining the absolute value of the difference of the voltage of a test QRS template and the voltage of a baseline QRS template at a plurality of sample points and detecting ischemia if the sum of the differences at the plurality of sample points is greater than an ischemia detection threshold. This known technique of monitoring ischemia is based upon the fact that an ischemic condition alters the depolarization and repolarization characteristics of the heart. For example, an ischemic region in the ventricle of the heart slows down the propagation of the excitation wave through the ventricles and is evidenced by changes in the QRS complex which models excitation wave propagation through the ventricles.

U.S. Pat. No. 7,610,086 relates to a system and a method for detecting cardiac ischemia in real-time using a pattern classifier implemented within an implanted medical device. Values representative of morphological features of electrical cardiac signals are detected by the implantable medical device. Then, a determination is made as to whether the patient is subject to an on-going episode of cardiac ischemia by applying the values to a pattern classifier configured to identify patterns representative of cardiac ischemia.

In U.S. Pat. No. 6,016,443 is disclosed a device that evaluates predetermined relations between sensed repolarization and sensed workload in order to identify a state of ischemia.

U.S. Pat. No. 7,657,309 relates to a method for measuring human heart muscle viability using myocardial electrical impedance. Implementations of the method are used to detect the extent of change of myocardial electrical impedance from a mean baseline value to provide diagnosis of the extent of ischemia, stenosis, tissue rejection, and reperfusion.

However, there is a need to achieve an improved measurement technique for determining ischemia risk.

### SUMMARY OF THE INVENTION

The above-mentioned object is achieved by the present invention according to the independent claims.

Preferred embodiments are set forth in the dependent claims.

Thus, the present invention relates to an implantable medical device comprising a coronary perfusion measurement unit adapted to measure and determine parameters related to coronary perfusion of heart tissue, said parameters include time periods and perfusion magnitudes.

The coronary perfusion measurement unit is configured to determine a time period T related to a perfusion event of a coronary vessel and including a reperfusion time period, where a perfusion event is defined as a decrease of coronary perfusion followed by reperfusion, and to generate a time period signal in dependence thereto. The implantable medical device further comprises a coronary flow calculation unit that is adapted to receive the time period signal and that is adapted to process the time period and to generate an ischemia risk indicating index I in dependence of the time period.

It is proposed, according to the present invention, a less exact measurement, but a possibility to evaluate the risk of imminent ischemia automatically, preferably using existing devices, without needing to involve highly trained specialists and hospital visits.

The following problems are solved by the present invention:

Many times ischemia is silent, i.e. the patient is not aware of his/her ischemia, and that ischemia may occur without earlier warning signs.

To evaluate the risk of ischemia today, as discussed in the background section, it is often required to put the patients in the catheterization lab or subject them to an exercise test, which is bothersome for the patient and expensive for the hospital.

The present invention relates to a fully automatic way of checking the risk for impending ischemia without any of those costly and invasive drawbacks.

The above problems are solved by a method and a device according to the present invention by:

Identifying a temporary reduction of coronary perfusion.

Monitoring the rate with which the myocardium is reperfused.

Creating an index representative of the risk of ischemia based upon the rate of reperfusion.

The basic principle behind the present invention is to detect a temporary reduction or worsening of the coronary perfusion and then measure the response to this reduction. There are several ways to induce and to detect the aforementioned reduction and also several ways of measuring the response. A number of them will be disclosed in the detailed description and it will also be described how the measurements are quantified and used as an ischemia risk notifier.

There are many advantages of the present invention:

The invention relates to a novel approach of determining an ischemia risk using existing technologies that does not necessarily require any additional decision making or managing by the physician—he/she will simply get either a trend or an alarm or just an additional tool at their disposal when assessing the patient's status.

It is easy to verify and to implement, and does not significantly influence the longevity of the battery of the implanted medical device.

The outcome of all these different areas of use, is that the patient's risk for developing ischemia can be measured in a relative way that has a high prognostic value and may be used to trend disease progression, make recommendations and tailor the medication according to what is best for any given patient.

### SHORT DESCRIPTION OF THE APPENDED DRAWINGS

FIG. 1 is a block diagram schematically illustrating an implantable medical device according to the present invention.

FIG. 2 is a graph illustrating the principle of the present invention.

FIG. 3 is a block diagram schematically illustrating a first embodiment of the implantable medical device according to the present invention.

FIG. 4 is a block diagram schematically illustrating a second embodiment of the implantable medical device according to the present invention.

FIG. 5 is a block diagram schematically illustrating a third embodiment of the implantable medical device according to the present invention.

FIG. 6 is a block diagram schematically illustrating a fourth embodiment of the implantable medical device according to the present invention.

FIG. 7 is a block diagram schematically illustrating another embodiment of the implantable medical device according to the present invention.

FIG. 8 is a flow diagram illustrating the method according to the present invention.

### DETAILED DESCRIPTION OF PREFERRED EMBODIMENTS OF THE INVENTION

The present invention will now be described with references to the accompanying drawings.

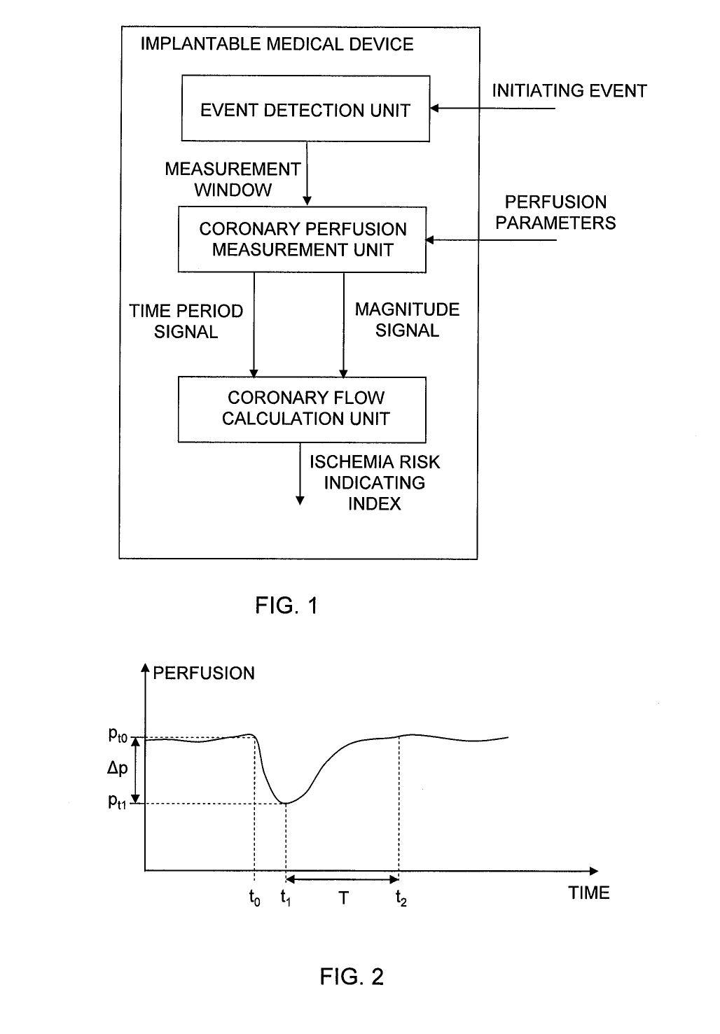

FIG. 1 shows a block diagram illustrating the present invention.

Thus, the present invention relates to an implantable medical device comprising a coronary perfusion measurement unit adapted to measure and determine parameters related to coronary perfusion of heart tissue, the parameters include time periods and perfusion magnitudes. The coronary perfusion measurement unit is configured to determine a time period T related to a perfusion event of a coronary vessel and including a reperfusion time period.

The perfusion event is defined as a decrease of coronary perfusion followed by reperfusion. The coronary perfusion measurement unit is configured to generate a time period signal in dependence of the determined time period T.

The implantable medical device further comprises a coronary flow calculation unit adapted to receive the time period signal. The coronary flow calculation unit is adapted to process the time period and to generate an ischemia risk indicating index I in dependence of the processed time period.

According to a preferred embodiment the time period T is determined as the time period that starts when the perfusion magnitude is at its minimum and ends when the perfusion magnitude is back to an initial perfusion level.

Within the scope of the invention as it is defined by the appended claims the interpretation of the time period T should be as comprising a major part of the reperfusion period and not necessarily starting exactly at the minimum perfusion magnitude but starting within a preset magnitude range from the minimum perfusion magnitude, e.g. 10% of the total perfusion magnitude difference from the minimum perfusion magnitude.

FIG. 2 is a graph illustrating the perfusion of a coronary vessel.

In FIG. 2 the Y-axis designates the perfusion level and the X-axis the time. The perfusion level has an initial perfusion level, essentially defined as the perfusion level which represents an initial coronary flow in a vessel, for example a non-constricted vessel. By initial level, or initial flow, is meant the level, or the flow, immediately before the perfusion event.

With references to FIG. 2 the start of the time period T is designated by t₁ and the end of the time period T is designated by t₂ and the time period T is then t₂-t₁. t₀ designates the start of a perfusion event. The initial perfusion level is designated by pₜ₀, and the minimum perfusion level is designated by pₜ₁.

According to a preferred embodiment the ischemia risk indicating index I is directly dependent upon time period T.

The length of the time period T is typically related to the heart cycle length, i.e. in the interval of 0.5-2.5 s.

The coronary perfusion measurement unit is also adapted to determine a perfusion magnitude measure Δp during a perfusion event, being the magnitude difference between an initial perfusion level and the minimum perfusion level, and to generate a magnitude signal that is applied to the coronary flow calculation unit. The perfusion magnitude measure Δp is shown in FIG. 2.

It is then possible to determine a normalized ischemia risk indicating index Iₙₒᵣₘ, which is determined as Iₙₒᵣₘ=T/Δp.

This ratio takes into consideration that depending on a number of physiological factors such as stress level, physical exertion, disease state, time of day, etc., the effect of an initiating event will be different in magnitude. By normalizing the time of recovery by the effect of the initiating event it is possible to adjust for that and obtain a fair trend of the ischemia risk indicating index I for a certain patient irrespective of the effect of each specific initiating event.

A schematic view of what the sensed signal and the response to the decreased perfusion look like are presented in FIG. 2.

The initiating event occurs before or at time instant t₀ and a result of that is a decline in coronary perfusion which is detected by the coronary perfusion measurement unit.

Time instant t₁ is defined as the time at which the perfusion magnitude is at its minimum. The time instant t₂ is defined as the point in time where the perfusion magnitude is back to where it was immediately prior to the initiating event. The maximum shift in the coronary perfusion (regardless of what unit or sensor signal it is) is referred to as Δp (or delta perfusion), defined as Δp=pₜ₀−pₜ₁, which is a measure of the resulting effect of the initiating event.

As discussed above a time period T is defined as: T=t₂−t₁, which roughly is the time for the tissue to get reperfused after the initiating event. The present invention utilizes the fact that the time period T will be prolonged as the vessels get narrower due to calcification and atherosclerosis. That is why this technique will serve as a valuable measure of at what risk a patient is of having ischemia or angina pectoris if the patient is exerted or other factors change.

It is possible to enhance the stability of this procedure by making the definitions of for instance t₂ more advanced. t₂ may then be defined as the time instant at which a window started, during which no recorded samples are more than e.g. 0.1 Δp below the initial perfusion value, or similar, to avoid temporary noisy samples reducing the fidelity of the system. These more advanced means of determining t₁ and t₂ are merely a matter of choice and are relatively straight forward and will not be dealt with further herein.

Thus, the time period T may essentially be defined as the time period of reperfusion, lasting from the peak perfusion (lowest level of perfusion) to the end of the reperfusion period.

In a preferred embodiment of the present invention the implantable medical device further comprises an event detection unit (see FIG. 1), whereby the event detection unit is arranged to detect an initiating event resulting in a perfusion event and is arranged to start a measurement window and activate the coronary perfusion measurement unit upon detection of an initiating event in order to determine the parameters related to the perfusion event. The length of a measurement window is naturally related to the time length of the entire perfusion event, i.e. essentially having a length of approximately 0.5-3.0 s.

According to a preferred embodiment the perfusion event is initiated by a premature ventricular contraction (PVC), which, according to one alternative is an intrinsic/spontaneous heart activity.

A PVC is defined as a ventricular event that follows immediately upon a previous ventricular event, without an atrial event in between.

A PVC is caused by an ectopic cardiac pacemaker located in the ventricle. PVCs are characterized by premature and bizarrely shaped QRS complexes usually wider than 120 ms. These complexes are not preceded by a P wave, and the T wave is usually large, and its direction is opposite the major deflection of the QRS.

In order to identify a PVC the event detection unit is adapted to sense electrical heart events.

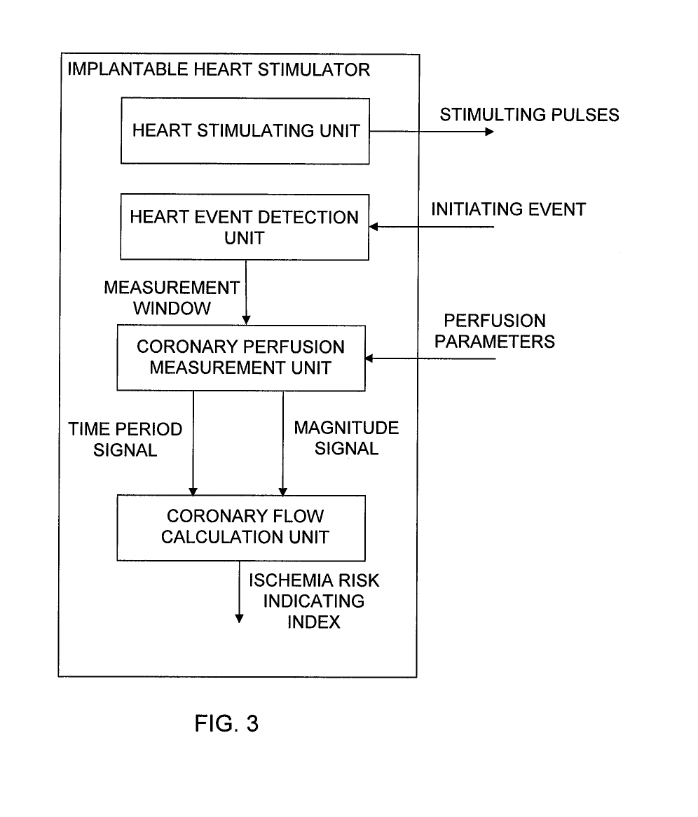

In a first embodiment, illustrated by FIG. 3, the implantable medical device is an implantable heart stimulator including a heart stimulating unit and the event detection unit is a heart event detection unit adapted to sense electrical heart events. By a heart stimulator is generally meant an implantable pacemaker, an implantable cardioverter defibrillator (ICD) or an implantable cardiac resynchronization therapy (CRT) device. The heart stimulating unit is adapted to generate and supply stimulation pulses (or defibrillation pulses) to heart tissue via one or more electrodes arranged at one or more electrode leads (not shown in the figures). Normally some or all of these electrodes are also used to sense electrical heart events.

According to an alternative embodiment the PVC is provoked by altering the stimulation regimen applied by the heart stimulating unit. Alternatively, the perfusion event may be provoked by creating an inter-ventricular dyssynchrony by applying a predefined pacing therapy, e.g. a rather short atrioventricular-delay (AV-delay) and a very long ventricular-ventricular-delay (VV-delay), by the heart stimulating unit.

Another possibility is to provoke the perfusion event by applying hemodynamically poor pacing settings, with for instance randomly varied RR in order to induce a reduced perfusion, by the heart stimulating unit.

In order to detect a spontaneous PVC, a provoked PVC, or an altered stimulation regimen, IEGM or ECG sensing and processing capabilities are required, which is embodied by the heart event detection unit, as well as ventricular pacing capabilities, which is embodied by the heart stimulating unit which is illustrated in FIG. 3.

The heart event detection unit is sensing and monitoring the IEGM or ECG and when the time has come to induce a temporary reduced perfusion one or more PVCs (premature ventricular contractions) are induced shortly after the T-wave and before the next P-wave. This will cause an unbalance of the local circulation and reduce the coronary perfusion immediately following the first PVC. The matter of determining whether one PVC or n

PVCs is the best choice would have to be determined empirically, but the principle still holds. Preferably one PVC is used. The efficacy of one PVC in reducing the perfusion has been confirmed.

There are some situations where provoking PVC may not be feasible. If the patient is suffering from chronic or persistent atrial fibrillation (AF), resulting in intermittent AV-conduction of high atrial rates resulting in greatly varied ventricular contraction frequency due to the intermittent conduction.

Another situation where PVC may be difficult to detect is for patients with sick sinus node syndrome since no intrinsic atrial activities are present.

This is also the case for patients who have undergone AV node ablation or suffer from AV-block III.

In those situations two main possibilities exist.

A: define a region of acceptable or normal RR-intervals. For instance a default setting could be that, given that the patient is in rest mode, an RR-interval <500 ms is to be classified as a hemodynamically poor contraction, much similar in its effects to a PVC. So if this happens the hemodynamic effect and subsequent reperfusion of that event, may be handled much in the same way that a PVC had been.

B: create the PVC-like condition by actively pacing the ventricle(s) prior to the next atrial event, or with an RR-interval that does not exceed the predefined limit (see A), depending on which the current situation is with respect to for example the activity level.

There exist naturally further methods of inducing a perfusion event.

One alternative way is to induce a perfusion event by occlusion of a vessel using a balloon catheter or similar.

Another alternative way is by inducing cold liquid through a special catheter.

Sensing the perfusion, and more importantly, the change thereof, can be done by using different sensor techniques.

A suitably placed oxygen sensor (like SᵥO₂ or pO₂), a local impedance measurement or use of a photoplethysmography technique (PPG) could divulge the desired information.

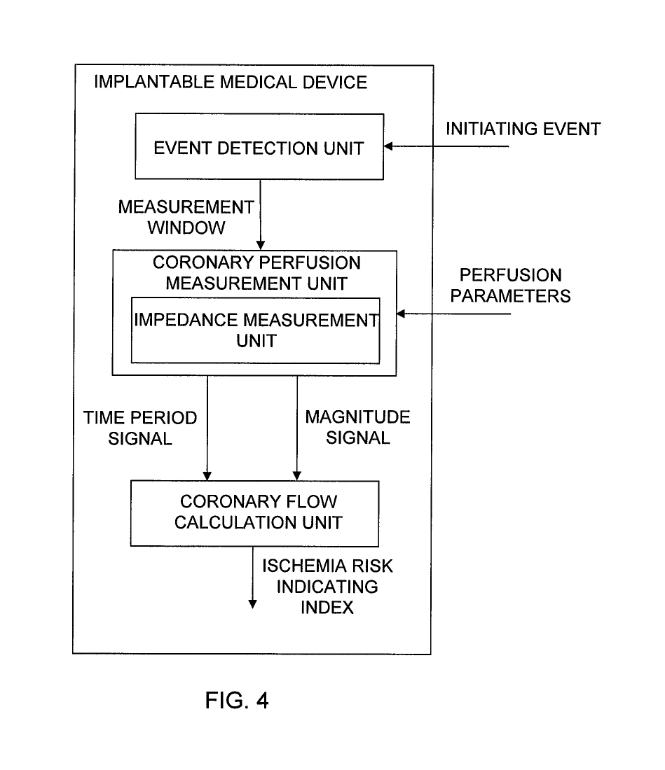

According to a second embodiment of the present invention the parameters related to blood perfusion is determined by local impedance measurements. This embodiment is illustrated by FIG. 4.

The coronary perfusion measurement unit then includes an impedance measurement unit adapted to measure and determine parameters related to blood perfusion of heart tissue.

The perfusion and reperfusion of the coronary vessels are directly dependent upon local electrical impedance values. The impedance measurement is typically performed by applying a measurement current between two electrodes arranged such that the electrical impedance between these electrodes, or other electrodes used as measurement electrodes, covers the coronary vessel which is subject for measurements.

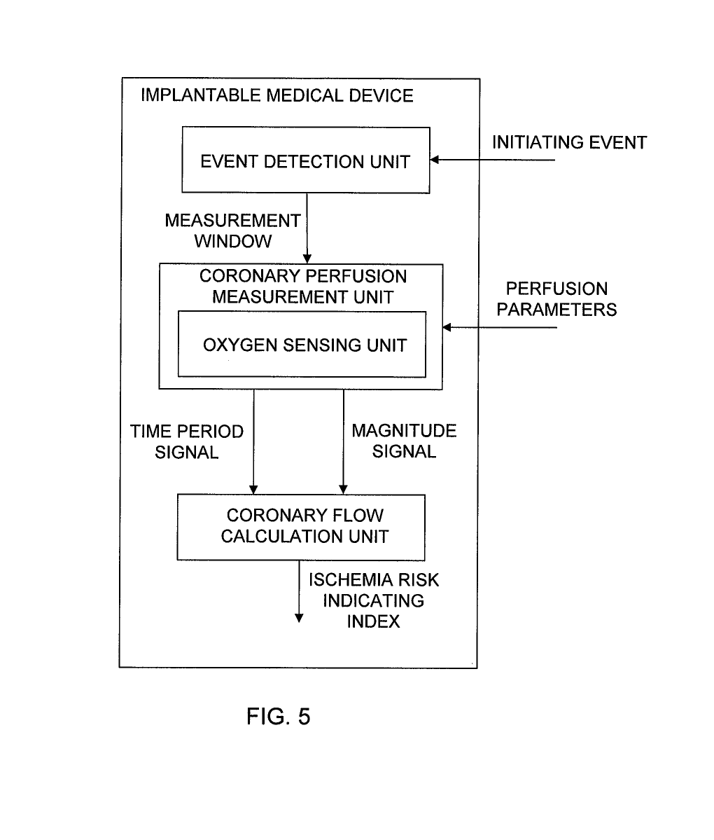

According to a third embodiment the coronary perfusion measurement unit includes an oxygen sensing unit adapted to measure and determine parameters related to blood perfusion of heart tissue. This embodiment is illustrated by FIG. 5. The oxygen sensing unit is adapted to determine SᵥO₂ or pO₂.

By measuring pO₂ locally in tissue it is possible to obtain information about e.g. circulation. This may be realized by using an electrode for pacemakers, based e.g. on the principle of pulsed polarography over a golden cathode.

Oxygen saturation (SᵥO₂)ᵥ may be measured by using an oxygen sensor portion of an electrode lead and provided with a sealed capsule containing red and infrared emitting diodes, a photo detector and an processing circuit. Oxygen saturation is then determined as the ratio of red/infrared light reflection.

The sensor used may ideally be placed anywhere on the epicardium or close enough so that the necessary measurement may be conducted. The positioning of the appropriate sensor on the coronary sinus lead (which targets the left ventricle) would also be a reasonable implementation.

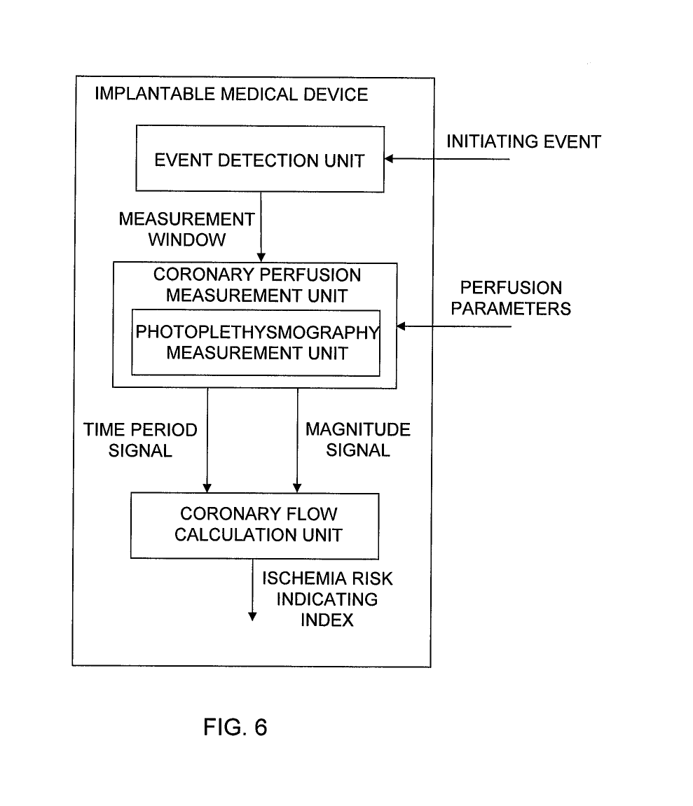

According to a fourth embodiment the coronary perfusion measurement unit includes a photoplethysmography measurement unit adapted to measure and determine parameters related to blood perfusion of heart tissue. This embodiment is illustrated by FIG. 6. That technique is based on one or more light emitting diodes (LEDs) that emit light and the reflected light is then measured. The amount of light that is absorbed by the surrounding tissue is proportional to the oxygen content of the tissue. Also in this embodiment, the sensor may ideally be placed anywhere on the epicardium or close enough so that the necessary measurement may be conducted. The positioning of the appropriate sensor on the coronary sinus lead (which targets the left ventricle) would also be a reasonable implementation.

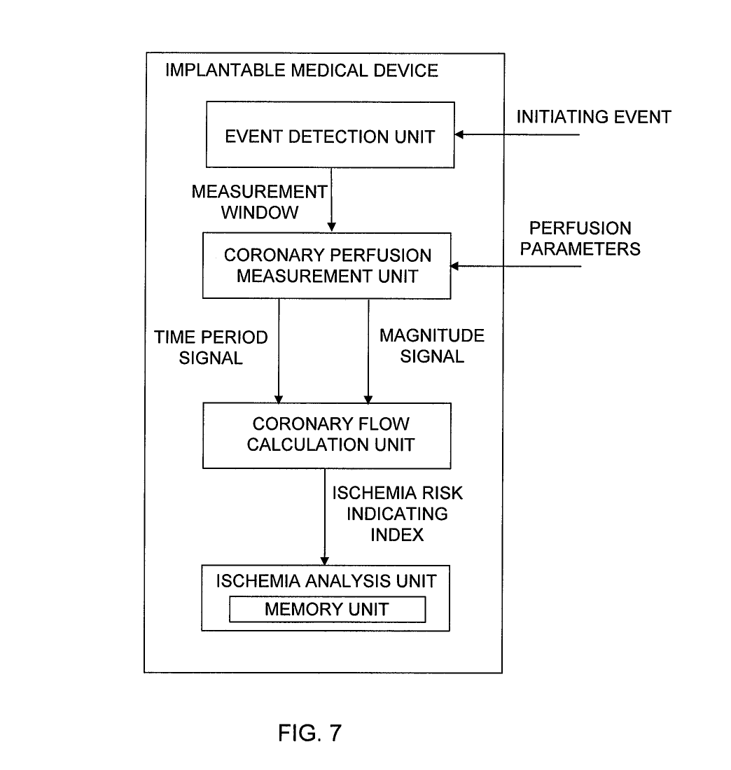

Preferably, the implantable medical device further comprises an ischemia analysis unit including a memory unit. This embodiment is illustrated by FIG. 7. The ischemia indicating index I is continuously determined by the coronary flow calculation unit and applied to the ischemia analysis unit where it is stored in said memory unit. Thus, the ischemia analysis unit is adapted to receive the ischemia risk indicating index and to store it in the memory unit. The analysis unit is configured to determine changes of stored ischemia risk indicating indexes in order to e.g. identify positive or negative trends of those changes.

The stored values may be transferred to an external device (not shown) by use of conventional telemetry and used for later evaluation. The ischemia analysis unit is naturally applicable for all previous embodiments.

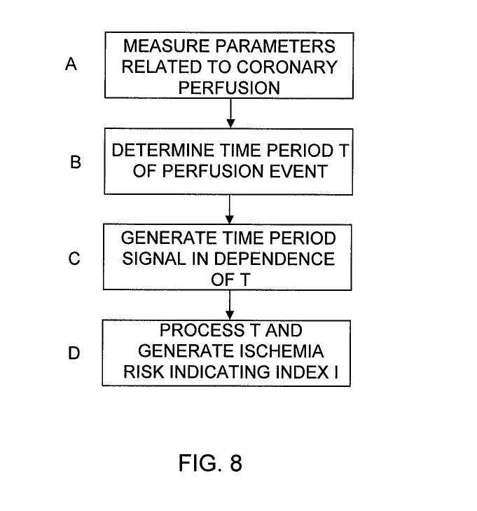

With references to FIG. 8 a method according to the present invention now will be described.

The present invention also relates to a method for determining an ischemia risk, comprising:

A) measuring and determining parameters related to coronary perfusion of heart tissue, said parameters include time periods and perfusion magnitudes;

B) determining a time period T related to a perfusion event of a coronary vessel and including a reperfusion time period, where a perfusion event is defined as a decrease of coronary perfusion followed by reperfusion,

C) generating a time period signal in dependence of said determined time period T;

D) processing said time period T and generating an ischemia risk indicating index I in dependence of said time period.

According to the method the time period T is preferably determined as the time period that starts when the perfusion magnitude is at its minimum and ends when the perfusion magnitude is back to an initial perfusion level. The definition of this level, and acceptable variations in relation to this level, have been discussed above.

The ischemia risk indicating index I is directly dependent upon the time period T.

In a further embodiment the method comprises determining a perfusion magnitude measure Δp during a perfusion event, being the magnitude difference between an initial perfusion level and the minimum perfusion level, and generating a magnitude signal in dependence thereto and applying it to a coronary flow calculation unit. A normalized ischemia risk indicating index Iₙₒₘ, is defined as Iₙₒᵣₘ=T/Δp.

In one embodiment the method comprises detecting an initiating event resulting in a perfusion event, and starting a measurement window and activating the coronary perfusion measurement unit upon detection of an initiating event in order to determine the parameters related to the perfusion event.

In one embodiment the initiating event is a spontaneous PVC. This embodiment is discussed above in relation to the description of the implantable medical device.

In another embodiment the perfusion event instead is a provoked event and the method comprises provoking a PVC, initiating the perfusion event, by altering a stimulation regimen applied to the heart tissue. Alternatively, the perfusion event may be provoked by creating an inter-ventricular dyssynchrony by applying predefined pacing therapy, e.g. a rather short AV-delay and a very long VV-delay, to the heart tissue.

As an alternative variation, the perfusion event is provoked by applying hemodynamically poor pacing settings, with for instance randomly varied RR in order to induce a reduced perfusion, to said heart tissue.

In one embodiment the method comprises measuring and determining parameters related to blood perfusion of heart tissue by performing impedance measurements.

In another embodiment the method comprises measuring and determining parameters related to blood perfusion of heart tissue by performing oxygen measurements, e.g. by determining SᵥO₂ or pO₂.

In yet another embodiment the method comprises measuring and determining parameters related to blood perfusion of heart tissue by performing photoplethysmography measurements.

These different methods of measuring and determining parameters related to blood perfusion of heart tissue are discussed in more detail above in connection with the description of the implantable medical device.

The method preferably comprises storing determined ischemia risk indicating index and determining changes of stored ischemia indicating indexes in order to identify positive or negative trends of those changes.

Furthermore, these stored values may be transmitted to an external device by using conventional telemetry.

The resulting device and method described herein for determining the ischemia risk indicating index may have several different applications. They can be used as a complement to a stress echo, or similar measurement technique, during a hospital visit where one I-value is obtained during rest and another during exercise, which may further enhance specificity. The method can also be programmed to be carried out automatically by the implantable medical device according to a pre-set schedule and alert if a sudden change of the measured I-value is detected. Still another possibility could be that a test is triggered by another event, e.g. an AF-episode, another tachycardia, changes in heart rate variability or rest- or breathing patterns. Another potential application would be to confirm the effects of a Percutaneous Coronary Intervention (PCI) in clinic.

It has already been mentioned that different physiological parameters can affect the result of the I-value. It is a possibility to also use inputs from other sensors, than those discussed above, which would allow us to either:
A) specify certain conditions that have to be met prior to acquiring the perfusion parameters, or
B) record anyway, but bin the data according to the different physiological parameters so that the comparisons and trends made afterwards will be valid.

The present invention is not limited to the above-described preferred embodiments. Various alternatives, modifications and equivalents may be used. Therefore, the above embodiments should not be taken as limiting the scope of the invention, which is defined by the appending claims.

1. An implantable medical device comprising:
a coronary perfusion measurement unit adapted to measure and determine parameters related to coronary perfusion of heart tissue, the parameters including time periods and perfusion magnitudes, wherein the coronary perfusion measurement unit is configured to determine a time period T related to a perfusion event of a coronary vessel and includes a reperfusion time period, wherein the perfusion event is defined as a decrease of coronary perfusion followed by reperfusion, and wherein the coronary perfusion measurement unit is configured to generate a time period signal in dependence thereto; and
a coronary flow calculation unit adapted to receive the time period signal and adapted to process the time period and generate an ischemia risk indicating index I in dependence of the time period.

2. The implantable medical device according to claim 1, wherein the time period T is determined as the time period that starts when the perfusion magnitude is at its minimum and ends when the perfusion magnitude is back to an initial perfusion level.

3. The implantable medical device according to claim 1, wherein the ischemia risk indicating index I is directly dependent upon the time period T.

4. The implantable medical device according to claim 1, wherein the coronary perfusion measurement unit is adapted to determine a perfusion magnitude measure Δp during a perfusion event and to generate a magnitude signal that is applied to the coronary flow calculation unit, wherein the perfusion magnitude measure Δp is the magnitude difference between an initial perfusion level and the minimum perfusion level.

5. The implantable medical device according to claim 4, wherein a normalized ischemia risk indicating index Iₙₒᵣₘ is determined as Iₙₒᵣₘ=T/Δp.

6. The implantable medical device according to claim 1, wherein the implantable medical device further comprises an event detection unit, the event detection unit is arranged to detect an initiating event resulting in a perfusion event and is arranged to start a measurement window and activate the coronary perfusion measurement unit, upon detection of an initiating event, in order to determine the parameters related to the perfusion event.

7. The implantable medical device according claim 1, wherein the perfusion event is initiated by a premature ventricular contraction (PVC).

8. The implantable medical device according to claim 7, wherein the PVC is an intrinsic/spontaneous heart activity.

9. The implantable medical device according to claim 1, wherein the medical device is a heart stimulator comprising a heart stimulating unit and a heart event detection unit adapted to sense electrical heart events.

10. The implantable medical device according to claim 1, wherein the perfusion event is initiated by a premature ventricular contraction (PVC), wherein the medical device is a heart stimulator comprising a heart stimulating unit and a heart event detection unit adapted to sense electrical heart events, and wherein the PVC is provoked by altering the stimulation regimen applied by the heart stimulating unit.

11. The implantable medical device according to claim 9, wherein the perfusion event is provoked by creating an inter-ventricular dyssynchrony by applying predefined pacing therapy, by the heart stimulating unit.

12. The implantable medical device according to claim 9, wherein the perfusion event is provoked by applying hemodynamically poor pacing settings in order to induce a reduced perfusion, by the heart stimulating unit.

13. The implantable medical device according to claim 1, wherein the coronary perfusion measurement unit comprises an impedance measurement unit adapted to measure and determine parameters related to blood perfusion of heart tissue.

14. The implantable medical device according to claim 1, wherein the coronary perfusion measurement unit comprises an oxygen sensing unit adapted to measure and determine parameters related to blood perfusion of heart tissue.

15. The implantable medical device according to claim 14, wherein the oxygen sensing unit is adapted to determine SᵥO₂ or pO₂.

16. The implantable medical device according to claim 1, wherein the coronary perfusion measurement unit comprises a photoplethysmography measurement unit adapted to measure and determine parameters related to blood perfusion of heart tissue.

17. The implantable medical device according to claim 1, wherein the device further comprises an ischemia analysis unit having a memory unit, the ischemia analysis unit adapted to receive the ischemia risk indicating index and to store it in the memory unit.

18. The implantable medical device according to claim 17, wherein the analysis unit is configured to determine changes of stored ischemia risk indicating indexes in order to identify positive or negative trends of those changes.

19. A method to determine an ischemia risk, comprising:
A) measuring and determining parameters related to coronary perfusion of heart tissue, the parameters include time periods and perfusion magnitudes;
B) determining a time period T related to a perfusion event of a coronary vessel and including a reperfusion time period, where the perfusion event is defined as a decrease of coronary perfusion followed by reperfusion,
C) generating a time period signal in dependence of the determined time period T;
D) processing the time period T and generating an ischemia risk indicating index I in dependence of the time period.

20. The method according to claim 19, wherein the time period T is determined as the time period that starts when the perfusion magnitude is at its minimum and ends when the perfusion magnitude is back to an initial perfusion level.

21. The method according to claim 19, wherein the ischemia risk indicating index I is directly dependent upon the time period T.

22. The method according to claim 19, comprising determining a perfusion magnitude measure Δp during a perfusion event, and generating a magnitude signal in dependence thereto and applying it to a coronary flow calculation unit, wherein the perfusion magnitude measure Δp is the magnitude difference between an initial perfusion level and the minimum perfusion level.

23. The method according to claim 22, comprising determining a normalized ischemia risk indicating index Iₙₒᵣₘ as Iₙₒᵣₘ=T/Δp.

24. The method according to claim 19, comprising detecting an initiating event resulting in a perfusion event, and starting a measurement window and activating a coronary perfusion measurement unit, upon detection of an initiating event, in order to determine the parameters related to the perfusion event.

25. The method according to claim 19, comprising provoking a premature ventricular contraction (PVC), initiating the perfusion event, by altering a stimulation regimen applied to the heart tissue.

26. The method according to claim 19, comprising creating an inter-ventricular dyssynchrony, provoking the perfusion event, by applying predefined pacing therapy to the heart tissue.

27. The method according to claim 19, comprising provoking the perfusion event by applying hemodynamically poor pacing settings in order to induce a reduced perfusion to the heart tissue.

28. The method according to claim 19, comprising measuring and determining parameters related to blood perfusion of heart tissue by performing impedance measurements.

29. The method according to claim 19, comprising measuring and determining parameters related to blood perfusion of heart tissue by performing oxygen measurements.

30. The method according to claim 19, comprising measuring and determining parameters related to blood perfusion of heart tissue by performing photoplethysmography measurements.

31. The method according to claim 19, comprising storing determined ischemia risk indicating index and determining changes of stored ischemia indicating indexes in order to identify positive or negative trends of those changes.
